(19) United States
(12) Patent Application Publication
(10) Pub. No.: US 2007/0156171 A1
(43) Pub. Date: Jul. 5, 2007

(54) Implant Grasper

(75) Inventors: Philipp Lang, Lexington, MA (US); Albert G. Burdulis JR., San Francisco, CA (US); Wolfgang Fitz, Sherborn, MA (US)

Correspondence Address: BROMBERG & SUNSTEIN LLP, 125 SUMMER STREET, BOSTON, MA, 02110-1618, US

(73) Assignee: CONFORMIS, INC., Foster City, CA (US)

(21) Appl. No.: 11/562,724

(22) Filed: Nov. 22, 2006

Related U.S. Application Data
(63) Continuation-in-part of application No. 10/997,407, filed on Nov. 24, 2004.
Application No. 10/997,407 is a continuation-in-part of application No. 10/752,438, filed on Jan. 5, 2004.
Application No. 10/752,438 is a continuation-in-part of application No. 10/724,010, filed on Nov. 25, 2003.
Application No. 10/724,010 is a continuation-in-part of application No. 10/305,652, filed on Nov. 27, 2002.
Application No. 10/305,652 is a continuation-in-part of application No. 10/160,667, filed on May 28, 2002.
(63) Continuation-in-part of application No. 10/681,750, filed on Oct. 7, 2003.
(60) Provisional application No. 60/739,479, filed on Nov. 23, 2005.
(60) Provisional application No. 60/293,488, filed on May 25, 2001.
(60) Provisional application No. 60/363,527, filed on Mar. 12, 2002.
(60) Provisional application No. 60/380,695, filed on May 14, 2002.
(60) Provisional application No. 60/380,692, filed on May 14, 2002.
(60) Provisional application No. 60/467,686, filed on May 2, 2003.
(60) Provisional application No. 60/416,601, filed on Oct. 7, 2002.

(51) Int. Cl. A61B 17/00

(52) U.S. Cl. 606/205

(57) ABSTRACT

A device for grasping an implant is provided. The implant has a first and second surface, with at least one of the first and seconds surfaces being non-planar. The device includes a first grasping element having a first grasping surface that conforms to the first surface of the implant, and a second grasping element having a second grasping surface conforming to the second surface of the implant. At least one of the first grasping element and the second grasping element can be moved to grasp the implant, such that the first grasping surface contacts the first surface of the implant and the second grasping surface contacts the second surface of the implant.

### CROSS REFERENCE TO RELATED APPLICATIONS

This application claims priority from U.S. provisional patent application 60/739,479 entitled “IMPLANT GRASPER,” filed Nov. 23, 2005.

This application is also a continuation-in-part of U.S. patent application Ser. No. 10/997,407 entitled “PATIENT SELECTABLE KNEE JOINT ARTHROPLASTY DEVICES, filed Nov. 24, 2004, which is a continuation-in-part of U.S. Ser. No. 10/752,438, filed Jan. 5, 2004 which is a continuation-in-part of U.S. application Ser. No. 10/724,010 filed Nov. 25, 2003 entitled “PATIENT SELECTABLE JOINT ARTHROPLASTY DEVICES AND SURGICAL TOOLS FACILITATING INCREASED ACCURACY, SPEED AND SIMPLICITY IN PERFORMING TOTAL AND PARTIAL JOINT ARTHROPLASTY,” which is a continuation-in-part of U.S. Ser. No. 10/305,652 entitled “METHODS AND COMPOSITIONS FOR ARTICULAR REPAIR,” filed Nov. 27, 2002, which is a continuation-in-part of U.S. Ser. No. 10/160,667, filed May 28, 2002, which in turn claims the benefit of U.S. Ser. No. 60/293,488 entitled “METHODS TO IMPROVE CARTILAGE REPAIR SYSTEMS”, filed May 25, 2001, U.S. Ser. No. 60/363,527, entitled “NOVEL DEVICES FOR CARTILAGE REPAIR, filed Mar. 12, 2002 and U.S. Ser. Nos. 60/380,695 and 60/380,692, entitled “METHODS AND COMPOSITIONS FOR CARTILAGE REPAIR,” and “METHODS FOR JOINT REPAIR,” filed May 14, 2002.

U.S. patent application Ser. No. 10/997,407 is also a continuation-in-part of U.S. application Ser. No. 10/681,750 filed Oct. 7, 2003 entitled “MINIMALLY INVASIVE JOINT IMPLANT WITH 3-DIMENSIONAL GEOMETRY MATCHING THE ARTICULAR SURFACES.”

U.S. patent application Ser. No. 10/997,407 also claims benefit of U.S. provisional patent application 60/467,686 filed May 2, 2003 entitled “JOINT IMPLANTS.”

This application is also a continuation-in-part of U.S. patent application Ser. No. 11/002,573, filed Dec. 2, 2004 entitled “Surgical Tools Facilitating Increased Accuracy, Speed and Simplicity in Performing Joint Arthroplasty.”

Each of these above-described applications is incorporated herein, in their entireties, by reference.

### TECHNICAL FIELD

The present invention relates to orthopedic methods, systems and devices, and more particularly, to a grasper device and method suitable for a joint prosthetic.

### BACKGROUND ART

Various prosthetic devices are often used during orthopedic surgery. For example, damaged cartilage in a knee joint may be treated by inserting an interpositional knee implant between the tibia and femur. Ensuring that the surfaces of such implants are not scratched or otherwise damaged when inserting or handling such implants is often critical to the overall success of the surgery.

However, implants grasped by conventional metal implant graspers run the risk of being damaged. This is particularly true when the implant is made of a softer material than the implant grasper, such as plastic. Compounding this problem is that various implants have non-planar concave or convex surfaces that do not conform with the grasping surfaces of the implant grasper. A poor grip on the implant may result in slippage and/or rotation of the implant. In addition to damaging the implant, such slippage and/or rotation makes insertion of the implant difficult for the surgeon.

### SUMMARY OF THE INVENTION

In accordance with one embodiment of the invention, a device for grasping an implant is provided. The implant has a first and second surface, with at least one of the first and seconds surfaces being non-planar. The device includes a first grasping element having a first grasping surface that substantially conforms to the first surface of the implant, and a second grasping element having a second grasping surface substantially conforming to the second surface of the implant. At least one of the first grasping element and the second grasping element can be moved to grasp the implant, such that the first grasping surface contacts the first surface of the implant and the second grasping surface contacts the second surface of the implant. Since the first and second grasping surfaces of the grasping device substantially conform with the first and second surfaces of the implant, the possibility of scratching or bending the implant is reduced and a firmer grip can advantageously be maintained.

In accordance with related embodiment of the invention, at least one of the first grasping element and the second grasping element is made of a metal, ceramic or a plastic. At least one of the first grasping element and the second grasping element may be a mold.

In accordance with another embodiment of the invention, a surgical instrument for grasping an implant having first and second surfaces is provided. The surgical instrument includes a first grasping element and a second grasping element. At least one of the first grasping element and the second grasping element can be moved to grasp the implant such that the first grasping element contacts the first surface of the implant and the second grasping element contacts the second surface of the implant. At least one of the first grasping element and the second grasping element is made of a pliable material.

In accordance with related embodiments of the invention, the pliable material may be a rubber or a plastic, such as polyethylene, PET or vinyl. At least one of the first grasping element and the second grasping element may be a mold.

In accordance with still another embodiment of the invention, an implant system is provided. The implant system includes an implant having first and second surfaces facing in substantially opposite directions. The first surface includes a first indentation. The system further includes an implant grasper for gripping the implant, the implant grasper including a first grasping element and a second grasping element. The first indentation has peripheral walls that prevent rotation of the implant when the first grasping element is received by the first indentation and the second grasping element of the implant grasper contacts the second surface to grasp the implant.

In related embodiments of the invention, the peripheral walls of the indentation form a v shape, and the first grasping element has peripheral walls that form a v shape. The implant may include a second indentation on the second surface, with the second grasping element shaped to be received by the second indentation such that rotation of the implant is prevented. The first grasping element may include an insertion protrusion, the first indentation including a protrusion receptacle for receiving the insertion protrusion. The protrusion receptacle may be a through hole.

In accordance with embodiments related to the above-described embodiments, the implant may be a joint implant, such as an interpositional implant. The implant may be for a hip, knee, shoulder, vertebrae, elbow, ankle, hand, foot or wrist. The first surface and the second surface of the implant may be a medial and lateral side wall, an anterior and posterior side wall, or a top and bottom wall, respectively. A first arm may include the first grasping element, and a second arm may include the second grasping element. The first arm and second arm may be pivotally attached. At least one of the first grasping element and the second grasping element may be integral or removably attached to the first arm and second arm, respectively.

In accordance with another embodiment of the invention, a method of grasping an implant is provided. The implant has a first and second surface, at least one of the first and seconds surfaces being non-planar. The method includes moving at least one of a first grasping element and a second grasping element to grasp the implant. The first grasping element includes a first grasping surface that substantially conforms to the first surface of the implant. The second grasping element includes a second grasping surface substantially conforming to the second surface of the implant. When grasping the implant, the first grasping surface and the second grasping surface contact the first surface and the second surface of the implant, respectively.

In accordance with another embodiment of the invention, a method for grasping an implant includes moving at least one of a first grasping element and a second grasping element to grasp the implant. At least one of the first grasping element and the second grasping element is made of a pliable material.

In accordance with yet another embodiment of the invention, a method for grasping an implant is provided. The implant has a first and second surface, the implant further including a first indentation on the first surface. The method includes moving at least one of a first grasping element and second grasping element to grasp the implant, such that the first grasping element is received by the first indentation, the first indentation including peripheral walls that prevent rotation of the first grasping element.

In accordance with related embodiments of the invention, the peripheral walls of the indentation form a v shape, and the first grasping element has peripheral walls that form a v shape. The implant may include a second indentation on the second surface, wherein moving includes placing the second grasping element into the second indentation. The second indentation may include peripheral walls that prevent rotation of the second grasping element. The first grasping element may include a protrusion, wherein the first indentation including a receptacle, and wherein moving includes inserting the protrusion into the receptacle. The protrusion may be, for example, an insertion pin. The receptacle may be a well, or a through-hole.

In accordance with related embodiments of the above-described methods, the implant may be a joint implant, such as an interpositional implant. The implant may be for a hip, knee, shoulder, vertebrae, elbow, ankle, hand, foot or wrist. The first grasping element may be removably attached to a first arm, and the second grasping element may be removably attached to a second arm. Moving at least one of a first grasping element and a second grasping element may include moving at least one of the first arm and the second arm. The first arm and the second arm may be pivotally attached. A mold may be used to form at least one of the first grasping surface and the second grasping surface. The implant may be inserted into a patient. Inserting the implant may include at least one of flexing, extending, rotating, abducting, and adducting the joint; and advancing the implant to the joint during, before or after said at least one of flexing, extending, rotating, abducting, and adducting.

In accordance with another embodiment of the invention, a method for grasping a joint implant is provided. The implant includes at least one weight-bearing surface made of a non-metallic material, and a first surface and a second surface made of metal. The method includes moving first and second grasping surfaces to grasp the implant, such that the first grasping surface and the second grasping surface contact the first surface and second surface of the implant, respectively. The first and second surfaces may not be weight-bearing surfaces.

In accordance with another embodiment of the invention, a kit includes an implant for use in a joint and having a first and second surface. At least one of the first and seconds surfaces is non-planar. The kit further includes a grasping device. The grasping device includes a first grasping element including a first grasping surface substantially conforming to the first surface of the implant, and a second grasping element including a second grasping surface substantially conforming to the second surface of the implant. At least one of the first grasping element and the second grasping element can be moved to grasp the implant, such that the first grasping surface contacts the first surface of the implant and the second grasping surface contacts the second surface of the implant.

In related embodiments of the invention, the implant may be an interpositional implant. The implant may be for a hip, knee, shoulder, vertebrae, elbow, ankle, hand, foot or a wrist.

In accordance with another embodiment of the invention, a kit includes an implant for a joint having a first and second surface. At least one of the first and seconds surfaces is non-planar. The kit further includes a surgical instrument having a first grasping element and a second grasping element. At least one of the first grasping element and the second grasping element can be moved to grasp the implant such that the first grasping element contacts the first surface of the implant and the second grasping element contacts the second surface of the implant. At least one of the first grasping element and the second grasping element is made of a pliable material.

In related embodiments of the invention, the implant may be an interpositional implant. The implant may be for a hip, knee, shoulder, vertebrae, elbow, ankle, hand, foot or a wrist.

### BRIEF DESCRIPTION OF THE DRAWINGS

The foregoing features of the invention will be more readily understood by reference to the following detailed description, taken with reference to the accompanying drawings, in which:

FIG. 1(a) is a perspective top view of an exemplary implant that may be grasped by a device, in accordance with one embodiment of the invention;

FIG. 1(b) is a cross-section taken along a sagittal plane in a body showing the implant of FIG. 1(a) implanted within a knee joint;

FIG. 1(c) is a cross-sectional view of an exemplary implant for a hip joint;

FIG. 2 shows a grasping device, in accordance with one embodiment of the invention;

FIG. 3 shows in more detail the grasping elements of the grasping device of FIG. 2, in accordance with one embodiment of the invention;

FIG. 4 shows the grasping device of FIG. 2 gripping an implant, in accordance with one embodiment of the invention;

FIG. 5 shows a cutaway view of the implant firmly contained by the grasping device of FIG. 2, in accordance with one embodiment of the invention;

FIG. 6 shows an implant system, in accordance with one embodiment of the invention;

FIG. 7 shows in more detail the implant of FIG. 6, in accordance with one embodiment of the invention; and

FIG. 8 shows in more detail the grasping elements of the implant grasper depicted in FIG. 6.

### DETAILED DESCRIPTION OF SPECIFIC EMBODIMENTS

In illustrative embodiments, devices and methods for grasping an implant are presented that reduce the risk of scratching or otherwise damaging the implant. Various embodiments prevent rotation and slippage of the implant when being grasped. Details are discussed below.

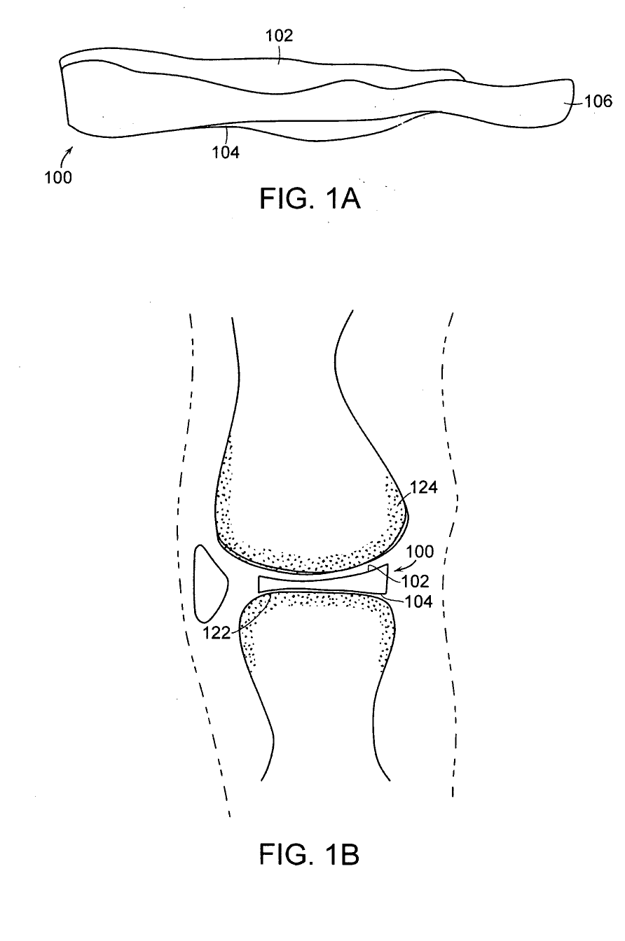

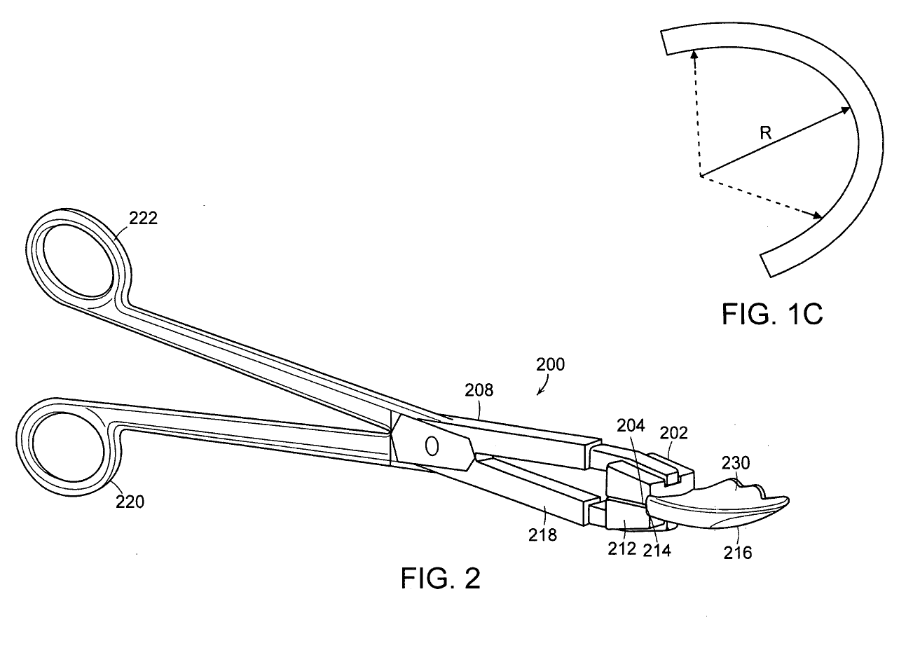

FIG. 1(a) is a slightly perspective top view of an exemplary implant 100 that may be grasped by a device, such as a surgical instrument, during an orthopedic procedure. The implant 100 is an interpositional knee implant, suitable for implantation at the tibial plateau of the knee joint, as described in U.S. patent application Ser. No. 10/997,407 entitled “Patient Selectable Knee Joint Arthroplasty Devices, filed Nov. 24, 2004, which is incorporated herein, it its entirety, by reference.

The implant 100 has an upper surface 102, a lower surface 104 and a peripheral edge 106. The upper surface 102 forms a mating surface for receiving the opposing joint surface (i.e., the upper surface 102 may substantially conform with, and be substantially a mirror image of, the opposing joint surface); in this instance partially concave to receive the femur. The concave surface may be variably concave such that it presents a surface to the opposing joint surface, e.g. a negative surface of the mating surface of the femur it communicates with. In various embodiments, the upper surface 102 may include a plurality of convexities and/or convexities.

The lower surface 104 has a convex surface that matches, or nearly matches, the tibial plateau of the joint such that it creates an anatomic or near anatomic fit with the tibial plateau. Depending on the shape of the tibial plateau, the lower surface can be partially convex as well. Thus, the lower surface 104 presents a surface to the tibial plateau that fits within the existing surface. It can be formed to substantially match the existing surface (i.e., the lower surface 104 may substantially conform with, and be substantially a mirror image of, the existing tibial plateau) or to match the surface after articular resurfacing.

As will be appreciated by those of skill in the art, the convex surface of the lower surface 204 need not be perfectly convex. Rather, the lower surface 204 more likely includes convex and concave portions that fit within the existing surface of the tibial plateau or the re-surfaced plateau. Thus, the surface may be essentially variably convex and concave.

FIG. 1(b) is a cross-section taken along a sagittal plane in a body showing the implant 100 implanted within a knee joint. The lower surface 104 of the implant 100 lies on the tibial plateau 122 and the femur 124 rests on the upper surface 102 of the implant 100.

In illustrative embodiments of the invention, a grasping device includes grasping surfaces that substantially conform with the non-planar surfaces of the implant 100. Since the grasping surfaces conform to the surfaces of the implant 100, a better grip is obtainable, and the risk of marring the surface of the implant 100 is advantageously reduced.

It is to be understood that the grasping device may be a surgical instrument customized for use with a wide variety of implants, including, without limitation, joint implants used in a hip (An exemplary implant for a hip joint is shown in FIG. 1(c). The radius r of this implant may be substantially constant when taken at any point along its length. The radius of the implant may be selected to approximate the radius of the femoral head that the implant is intended to correct and can be measured to an interior surface of the implant that engage the femoral head. Alternatively, the radius of the implant can be selected to approximate the radius of the acetabulum or a combination thereof. The radius of the interior surface of the implant faces the femur and can also match the radius of the femur or be similar to the radius of the acetabulum; the radius of the implant surface facing the acetabulum can also match that of the acetabulum or be similar to that of the femur), knee, shoulder, vertebrae, elbow, ankle, hand, foot and wrist. In various embodiments, the joint implant may be an interpositional joint implant, a cartilage defect conforming implant, a cartilage projected implant, and/or a subchondral bone conforming implant.

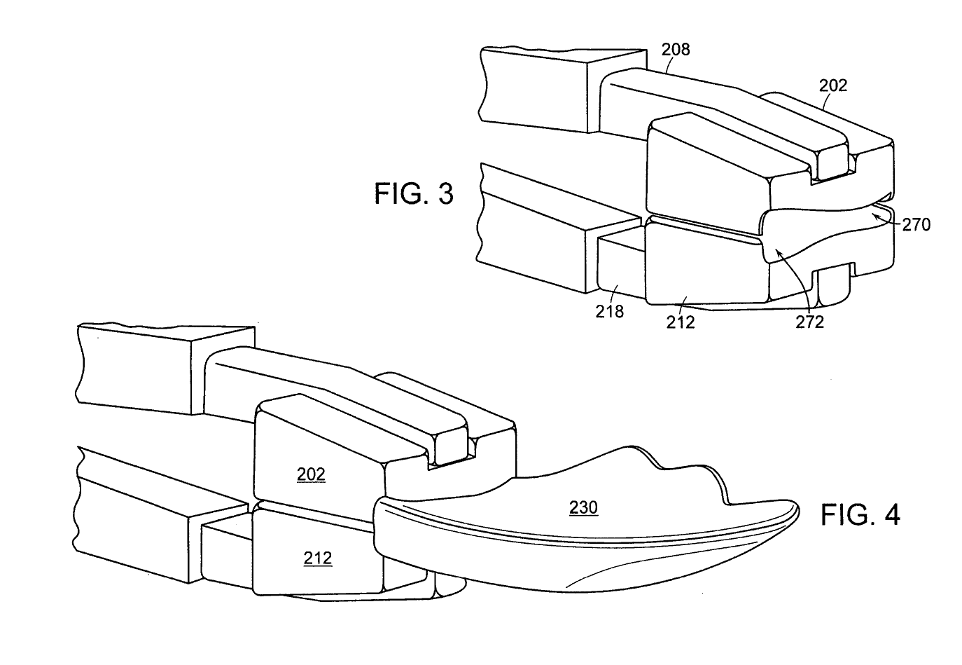

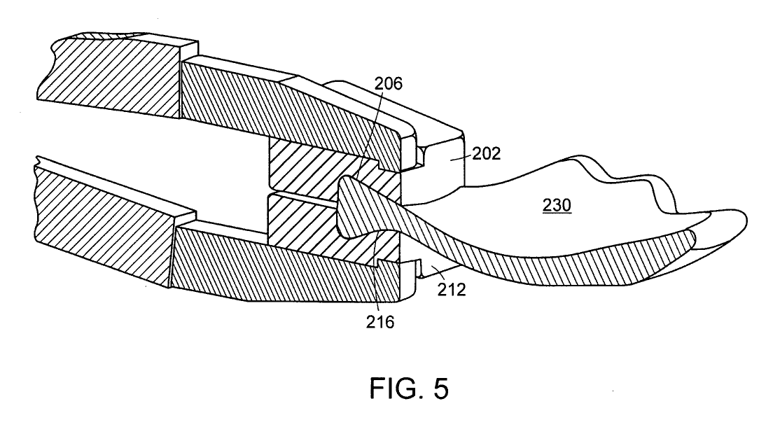

FIG. 2 shows a grasping device 200, in accordance with one embodiment of the invention. The grasping device 200 includes a first grasping element 202 and a second grasping element 212. The first grasping element 202 has a first grasping surface 204 that substantially conforms to a first surface 206 (see FIG. 5) of an implant 230. The second grasping element 212 has a second grasping surface 214 that substantially conforms to a second surface 216 (see FIG. 5) of the implant 230.

At least one of the first grasping element 202 and the second grasping element 212 can be moved to grasp the implant 230, such that the first grasping surface 204 contacts the first surface 206 of the implant 230 and the second grasping surface 214 contacts the second surface 216 of the implant 230. For example, the first grasping element 202 may be attached to a distal end of a first arm 208, with the second grasping element 212 attached to a distal end of a second arm 218. The first and second arms 208 and 210 may be pivotally attached to each other. Handles 220 and 222 at proximal ends of the first arm 208 and second arm 218 may be provided, respectively. In preferred embodiments, the handles are sufficiently sized so as to allow a surgeon to hold and manipulate the grasping device 200 with minimal effort.

In various embodiments, the first surface and the second surface of the implant 230 may be, without limitation, a top surface, a bottom surface, or a peripheral side of the implant 230. The grasper device 200 may grasp, for example, top and bottom surfaces of the implant 230; medial and lateral walls of the implant 230; or anterior and posterior walls of the implant 230.

FIG. 3 shows a detailed view of the grasping elements 202 and 212 of the grasping device 200 shown in FIG. 2. In various embodiments, the first grasping element 202 and/or the second grasping element 212 are removably attached to the first arm 208 and second arm 218, respectively. The capability to change grasping elements advantageously allows the grasping device 200 to be adapted to a plurality of implants that may be of different sizes and/or have varying surfaces. The grasping elements 202 and 212 may, without limitation, snap or slide onto arms 208 and 218, respectively. In other embodiments, the first grasping element 202 and/or the second grasping element 212 are integral to the first arm 208 and second arm 218, respectively.

In preferred embodiments, the grasping device 200 is made of biocompatible materials that do not cause inflammations or reactions when contacting a patient. The grasping elements 202 and 212 may be made of, without limitation, metal, ceramic, and/or various polymers/plastics. The grasping surfaces 204 and 214 of grasping elements 202 and 210, respectively, may be, without limitation, molded or machined to conform to the surfaces of the implant 230. As shown in FIG. 3, the grasping elements 202 and 212 may include cavities 270 and 272 having surfaces that conform to portions of the implant 230.

A wide-variety of metals are useful in the practice of the present invention, and can be selected based on any criteria. For example, material selection can be based on resiliency to impart a desired degree of rigidity. Non-limiting examples of suitable metals include silver, gold, platinum, palladium, iridium, copper, tin, lead, antimony, bismuth, zinc, titanium, cobalt, stainless steel, nickel, iron alloys, cobalt alloys, such as Elgiloy®, a cobalt-chromium-nickel alloy, and MP35N, a nickel-cobalt-chromium-molybdenum alloy, and Nitinol™, a nickel-titanium alloy, aluminum, manganese, iron, tantalum, crystal free metals, such as Liquidmetal® alloys (available from LiquidMetal Technologies, www.liquidmetal.com), and combinations thereof.

Suitable synthetic polymers include, without limitation, polyamides (e.g., nylon), polyesters, polystyrenes, polyacrylates, vinyl polymers (e.g., polyethylene, polytetrafluoroethylene, polypropylene and polyvinyl chloride), polycarbonates, polyurethanes, poly dimethyl siloxanes, cellulose acetates, polymethyl methacrylates, polyether ether ketones, ethylene vinyl acetates, polysulfones, nitrocelluloses, similar copolymers and mixtures thereof. Bioresorbable synthetic polymers can also be used such as dextran, hydroxyethyl starch, derivatives of gelatin, polyvinylpyrrolidone, polyvinyl alcohol, poly[N-(2-hydroxypropyl) methacrylamide], poly(hydroxy acids), poly(epsilon-caprolactone), polylactic acid, polyglycolic acid, poly(dimethyl glycolic acid), poly(hydroxy butyrate), and similar copolymers can also be used.

Other materials would also be appropriate, for example, the polyketone known as polyetheretherketone (PEEK™). This includes the material PEEK 450G, which is an unfilled PEEK approved for medical use available from Victrex of Lancashire, Great Britain. (Victrex is located at www.matweb.com or see Boedeker www.boedeker.com). Other sources of this material include Gharda located in Panoli, India (www.ghardapolymers.com).

The materials can be prepared by any of a variety of approaches including, for is example, injection molding, which is suitable, without limitation, for the production of polymer components with significant structural features, and rapid prototyping approaches, such as reaction injection molding and stereo-lithography. The substrate can be textured or made porous by either physical abrasion or chemical alteration to facilitate incorporation of, for example, a metal coating. Other processes are also appropriate, such as extrusion, injection, compression molding and/or machining techniques.

In various embodiments, the first grasping element 202 and/or the second grasping element 212 may include a pliable material such that grasping surfaces 204 and/or 214 is easily shaped to conform to surfaces 206 and/or 208 upon grasping the implant 230, respectively. The pliable material may be, without limitation, a rubber or plastic, such as polyethylene, PET or vinyl.

FIG. 4 shows a detailed view of the grasping device 200 gripping the implant 230, in accordance with one embodiment of the invention. FIG. 5 shows a cutaway view of the implant firmly contained by grasping elements 202 and 212, in accordance with one embodiment of the invention.

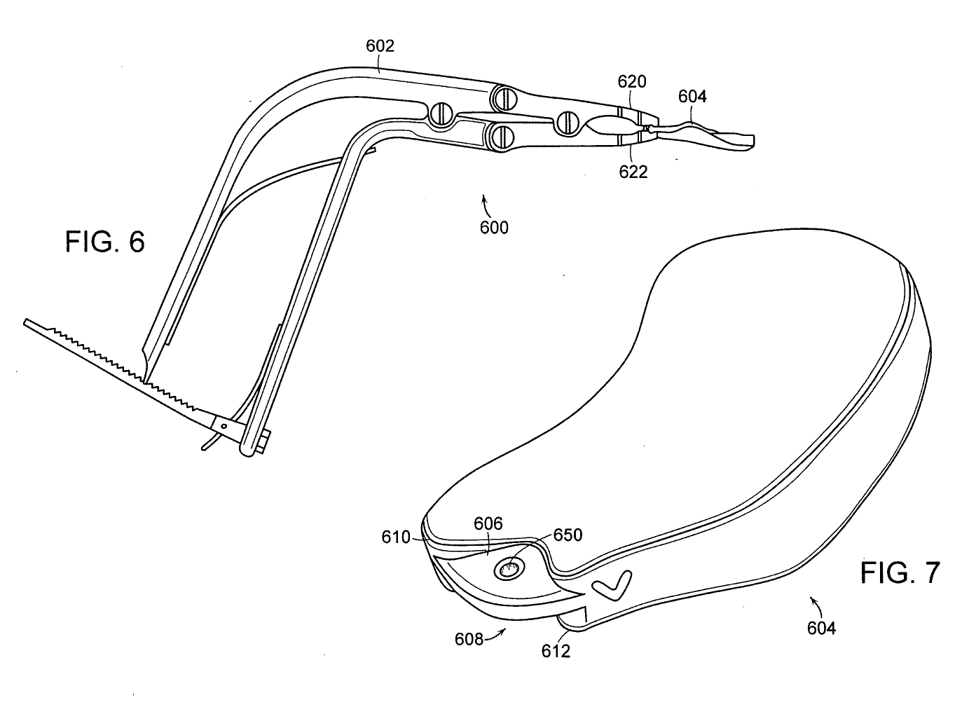

FIG. 6 shows an implant system 600, in accordance with one embodiment of the invention. The implant system 600 includes an implant grasper 602 having first and second grasping elements 620 and 622 for grasping an implant 604. The implant 602, shown in more detail in FIG. 7, includes indentations 606 and 608 on surfaces 610 and 612 of the implant 604, respectively. In alternative embodiments, only one of the surfaces 610 and 612 may have an indentation. The two surfaces 610 and 612 face in substantially opposite directions such that they can be grasped by the implant grasper 602. The indentations 606 and 608 have peripheral walls that prevent rotation of the implant 600 when grasped by grasping elements 620 and 622 of grasper 602.

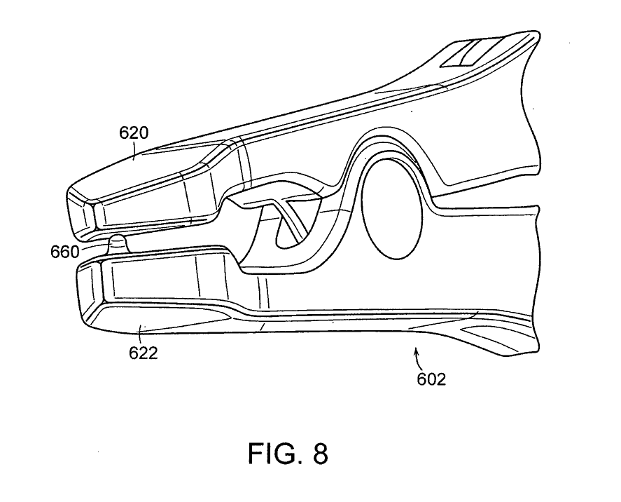

FIG. 8 shows the grasping elements 620 and 622 of implant grasper 602 in more detail. The peripheral walls of each grasping element 620 and 622 form a v shape. The peripheral walls of each indentation 606 and 608 also form a v shape, as shown in FIG. 7. Upon receiving the grasping elements 620 and 622, the peripheral walls of indentations 606 and 608 contact the peripheral walls of grasping elements 620 and 622, respectively, to substantially prevent rotation of the implant 604. It is to be understood that the peripheral walls of the indentations and grasping elements are not limited to a v shape, any number of shapes for the indentations may be utilized as long as rotation of the implant is prevented upon grasping.

In various embodiments, the indentation(s) on surfaces 610 and 612 of the implant 604 are not weight-bearing surfaces of the implant 604. The term “weight-bearing” as used herein means refers to the contact area between two opposing articular surfaces during activities of normal daily living. The term “articular” refers to any joint. The term “articular surface” refers to a surface of an articulating bone that is covered by cartilage. For example, in a knee joint several different articular surfaces are present, e.g. in the patella, the medial femoral condyle, the lateral femoral condyle, the medial tibial plateau and the lateral tibial plateau.

In various embodiments, the indentation 606 may include a receptacle 650 for accepting a protrusion 660 on the first grasping element 620. Upon the implant grasper 602 grasping the implant 604, the protrusion 660 inserted into the receptacle 650 helps to maintain the implant's 604 position between the first and second grasping element 620 and 622. The receptacle 650 may be a well, or a through-hole that extends between the first and second surfaces 610 and 612 of the implant 604. The protrusion 660 may be, for example, an insertion pin.

In the above-described embodiments, the implant may include several surfaces, including at least one weight-bearing surface, that are made of a non-metallic material. To avoid scratching or otherwise damaging the implant, the surfaces of the implant to be grasped by the implant grasper may be made of a metal, in accordance with various embodiments of the invention. The first and second grasping surfaces may be moved to grasp the implant, such that the first grasping surface and the second grasping surface contact first and second metallic surfaces the implant, respectively. In preferred embodiments, the metal surfaces may be positioned on non weight-bearing surfaces of the implant.

The grasping device in the above-described embodiments can be used to insert an implant into a joint of a patient. Inserting the implant into the patient may include flexing, extending, rotating, abducting, and adducting the joint. The implant may be advanced into to the joint during, before or after the flexing, extending, rotating, abducting, and adducting. Both the grasping device and the implant may be provided in a kit.

Although various exemplary embodiments of the invention have been disclosed, it should be apparent to those skilled in the art that various changes and modifications can be made which will achieve some of the advantages of the invention without departing from the true scope of the invention. These and other obvious modifications are intended to be covered by the appended claims.

What is claimed is:

1. A device for grasping an implant, the implant for use in a joint and having a first and second surface, at least one of the first and seconds surfaces being non-planar, the grasper device comprising:
a first grasping element including a first grasping surface substantially conforming to the first surface of the implant;
a second grasping element including a second grasping surface substantially conforming to the second surface of the implant;
wherein at least one of the first grasping element and the second grasping element can be moved to grasp the implant, such that the first grasping surface contacts the first surface of the implant and the second grasping surface contacts the second surface of the implant.

2. The device according to claim 1, further comprising a first arm that includes the first grasping element, and a second arm that includes the second grasping element.

3. The device according to claim 2, wherein the first arm and second arm are pivotally attached.

4. The device according to claim 2, wherein at least one of the first grasping element and the second grasping element is integral to the first arm and second arm, respectively.

5. The device according to claim 2, wherein at least one of the first grasping element and the second grasping element is removably attached to the first arm and second arm, respectively.

6. The device according to claim 1, wherein at least one of the first grasping element and the second grasping element is made of a material from the group of materials consisting of metal, ceramic and plastic.

7. The device according to claim 1, wherein at least one of the first grasping element and the second grasping element is a mold.

8. The device according to claim 1, wherein the implant is an interpositional implant.

9. The device according to claim 8, wherein the implant is for one of a hip, knee, shoulder, vertebrae, elbow, ankle, hand, foot and wrist.

10. The device according to claim 1, wherein the first surface and the second surface of the implant are one of a medial and lateral side wall, an anterior and posterior side wall, and a top and bottom wall, respectively.

11. A surgical instrument for grasping an implant having a first and second surface, the implant for use in a joint, at least one of the first and seconds surfaces being non-planar, the surgical instrument comprising:
a first grasping element;
a second grasping element;
wherein at least one of the first grasping element and the second grasping element can be moved to grasp the implant such that the first grasping element contacts the first surface of the implant and the second grasping element contacts the second surface of the implant, and wherein at least one of the first grasping element and the second grasping element is made of a pliable material.

12. The surgical instrument according to claim 11, wherein the surgical instrument further includes a first arm that includes the first grasping element, and a second arm that includes the second grasping element.

13. The surgical instrument according to claim 12, wherein the first arm and second arm are pivotally attached.

14. The surgical instrument according to claim 12, wherein at least one of the first grasping element and the second grasping element is integral to the first arm and second arm, respectively.

15. The surgical instrument according to claim 12, wherein at least one of the first grasping element and the second grasping element is removably attached to the first arm and second arm, respectively.

16. The surgical instrument according to claim 11, wherein the pliable material is one of a plastic and rubber.

17. The surgical instrument according to claim 11, wherein at least one of the first grasping element and the second grasping element is a mold.

18. The surgical instrument according to claim 11, wherein the implant is an interpositional implant.

19. The surgical instrument according to claim 11, wherein the implant is for one of a hip, knee, shoulder, vertebrae, elbow, ankle, hand, foot and wrist.

20. The surgical instrument according to claim 11, wherein the first surface and the second surface of the implant are one of a medial and lateral side wall, an anterior and posterior side wall, and a top and bottom wall, respectively.

21. An implant system comprising:
an implant having first and second surfaces facing in substantially opposite directions, the first surface including a first indentation; and
an implant grasper for gripping the implant, the implant grasper including a first grasping element and a second grasping element;
wherein the first indentation has peripheral walls that prevent rotation of the implant when the first grasping element is received by the first indentation and the second grasping element of the implant grasper contacts the second surface to grasp the implant.

22. The implant system according to claim 21, wherein the peripheral walls of the indentation form a v shape, and wherein the first grasping element has peripheral walls that form a v shape.

23. The implant system according to claim 21, wherein the first indentation is not a weight-bearing surface of the implant.

24. The implant system according to claim 21, further comprising a second indentation on the second surface of the implant, the second indentation for receiving the second grasping element of the implant grasper.

25. The implant system according to claim 24, wherein the second indentation includes peripheral edges that prevent rotation of the implant when the second grasping element is received by the second indention.

26. The implant system according to claim 21, wherein the first indentation further includes a receptacle for accepting a protrusion on the first grasping element.

27. The implant system according to claim 21, wherein the implant is an interpositional implant.

28. The implant system according to claim 21, wherein the implant is for one of a hip, knee, shoulder, vertebrae, elbow, ankle, hand, foot and wrist.

29. A method of grasping an implant, the implant for use in a joint and having a first and second surface, at least one of the first and seconds surfaces being non-planar, the method comprising:
moving at least one of a first grasping element and a second grasping element to grasp the implant, the first grasping element including a first grasping surface that substantially conforms to the first surface of the implant, the second grasping element including a second grasping surface substantially conforming to the second surface of the implant, wherein the first grasping surface and the second grasping surface contact the first surface and the second surface of the implant, respectively.

30. The method according to claim 29, further comprising:
removably attaching the first grasping element to a first arm.

31. The method according to claim 30, further comprising:
removably attaching the second grasping element to a second arm, wherein moving at least one of a first grasping element and a second grasping element includes moving at least one of the first arm and the second arm.

32. The method according to claim 31, wherein the first arm and the second arm are pivotally attached.

33. The method according to claim 29, further comprising:
using a mold to form at least one of the first grasping surface and the second grasping surface.

34. The method according to claim 29, further comprising:
inserting said implant into a patient.

35. The method according to claim 34, wherein inserting the implant includes:
at least one of flexing, extending, rotating, abducting, and adducting the joint; and
advancing the implant to the joint during, before or after said at least one of flexing, extending, rotating, abducting, and adducting.

36. The method according to claim 29, wherein the implant is an interpositional implant.

37. The method according to claim 29, wherein the implant is for one of a hip, knee, shoulder, vertebrae, elbow, ankle, hand, foot and wrist.

38. The method according to claim 29, wherein the first surface and the second surface of the implant are one of a medial and lateral side wall, an anterior and posterior side wall, and a top and bottom wall, respectively.

39. A method for grasping an implant, the implant for use in a joint, the method comprising:
moving at least one of a first grasping element and a second grasping element to grasp the implant, at least one of the first grasping element and the second grasping element made of a pliable material.

40. The method according to claim 39, further comprising:
removably attaching the first grasping element to a first arm.

41. The method according to claim 40, further comprising:
removably attaching the second grasping element to a second arm, wherein moving at least one of a first grasping element and a second grasping element includes moving at least one of the first arm and the second arm.

42. The method according to claim 41, wherein the first arm and the second arm are pivotally attached.

43. The method according to claim 39, further comprising:
inserting said implant into a patient.

44. The method according to claim 39, wherein inserting the implant includes:
at least one of flexing, extending, rotating, abducting, and adducting the joint; and
advancing the implant to the joint during, before or after said at least one of flexing, extending, rotating, abducting, and adducting.

45. The method according to claim 39, wherein the implant is an interpositional implant.

46. The method according to claim 39, wherein the implant is for one of a hip, knee, shoulder, vertebrae, elbow, ankle, hand, foot and wrist.

47. A method for grasping an implant, the implant having a first and second surface, the implant further including a first indentation on the first surface, the method comprising:
moving at least one of a first grasping element and second grasping element to grasp the implant, such that the first grasping element is received by the first indentation, the first indentation including peripheral walls that prevent rotation of the first grasping element.

48. The method according to claim 47, wherein the peripheral walls of the indentation form a v shape, and the first grasping element has peripheral walls that form a v shape.

49. The method according to claim 47, wherein the implant includes a second indentation on the second surface of the implant, and moving includes placing the second grasping element into the second indentation, the second indentation including peripheral walls that prevent rotation of the second grasping element.

50. The method according to claim 47, wherein the first grasping element includes an protrusion, wherein the first indentation including a receptacle, and wherein moving includes inserting the protrusion into the receptacle.

51. A method for grasping an implant, the implant for use in a joint, the implant including at least one weight-bearing surface made of a non-metallic material, and a first surface and a second surface made of metal, the method comprising:
moving first and second grasping surfaces to grasp the implant, such that the first grasping surface and the second grasping surface contact the first surface and second surface of the implant, respectively.

52. The method according to claim 51, wherein the first and second surfaces are not weight-bearing surfaces.

53. A kit comprising:
an implant for use in a joint and having a first and second surface, at least one of the first and seconds surfaces being non-planar; and
a grasping device including:
a first grasping element including a first grasping surface substantially conforming to the first surface of the implant; and
a second grasping element including a second grasping surface substantially conforming to the second surface of the implant;
wherein at least one of the first grasping element and the second grasping element can be moved to grasp the implant, such that the first grasping surface contacts the first surface of the implant and the second grasping surface contacts the second surface of the implant.

54. The kit according to claim 53, wherein the implant is an interpositional implant.

55. The kit according to claim 53, wherein the implant is for one of a hip, knee, shoulder, vertebrae, elbow, ankle, hand, foot and wrist.

56. A kit comprising:
an implant having a first and second surface, the implant for use in a joint, at least one of the first and seconds surfaces being non-planar; and
a surgical instrument comprising:
a first grasping element; and
a second grasping element;
wherein at least one of the first grasping element and the second grasping element can be moved to grasp the implant such that the first grasping element contacts the first surface of the implant and the second grasping element contacts the second surface of the implant, and wherein at least one of the first grasping element and the second grasping element is made of a pliable material.

57. The kit according to claim 56, wherein the implant is an interpositional implant.

58. The kit according to claim 56, wherein the implant is for one of a hip, knee, shoulder, vertebrae, elbow, ankle, hand, foot and wrist.
